(19) United States
(12) Patent Application Publication
(10) Pub. No.: US 2008/0132724 A1
(43) Pub. Date: Jun. 5, 2008

(54) Allophanate modified isocyanates which contain reactive unsaturation

(75) Inventor: Rick L. Adkins, Hurricane, WV (US)

Correspondence Address: BAYER MATERIAL SCIENCE LLC, 100 BAYER ROAD, PITTSBURGH, PA, 15205, US

(73) Assignee: Bayer MaterialScience LLC

(21) Appl. No.: 11/633,242

(22) Filed: Dec. 4, 2006

(51) Int. Cl. C07C 265/12; C07C 263/16

(52) U.S. Cl. 560/336; 560/359

(57) ABSTRACT

This invention relates to a novel liquid, storage-stable diphenylmethane diisocyanate which contains allophanate and reactive unsaturation. These novel storage-stable liquids comprise the reaction product of diphenylmethane diisocyanate with an ethylenically unsaturated alcohol, in the presence of an allophanate catalyst and a suitable inhibitor.

### BACKGROUND OF THE INVENTION

This invention relates to novel liquid allophanate-modified diphenylmethane diisocyanate compositions and to a process for the preparation of these novel liquid modified diisocyanate compositions.

Allophanate-modified diphenylmethane diisocyanates and prepolymers thereof are known and described in, for example, U.S. Pat. Nos. 5,319,053, 5,319,054, 5,440,003, 5,663,272 and 5,686,042. U.S. Pat. No. 5,319,053 discloses a process for preparing stable liquid allophanate-modified MDI isocyanates and prepolymers of these allophanate-modified MDI isocyanates. This process comprises reacting a specific MDI isomer composition with an aliphatic alcohol containing between 1 and 36 carbon atoms to give an allophanate-modified MDI having an NCO group content of 12 to 32.5%.

U.S. Pat. No. 5,319,054 describes a process for producing diallophanate-modified MDI by reacting the diurethane which is the reaction product of one equivalent of a diisocyanate with one equivalent of an aliphatic alcohol or an aromatic alcohol, with 4,4′-MDI containing about 2 to about 60% by weight of the 2,4-isomer of MDI. These diallophanates have NCO group contents of about 12 to about 30%.

EP 641,812 discloses the use of allophanate-modified MDI prepolymers in automotive RIM and rigid foam applications. It is a distinct feature of the invention that the flex modulus of the RIM products can be increased without modifying the chain extender composition or content.

Stable, liquid, diallophanate-modified diphenylmethane diisocyanate is known and described in, for example, U.S. Pat. No. 5,686,042. These diallophanate-modified diisocyanates are prepared from the diurethane of an alcohol and a diisocyanate and MDI. The diallophanate or a prepolymer thereof are disclosed as being used in a RIM process to produce elastomers with improved flex modulus. Since these diallophanates form elastomers with improved flex modulus, lower levels of fast reacting chain extenders are required to reach a given stiffness.

Urethane-free, allophanate-modified diphenylmethane diisocyanates are known and disclosed in, for example, U.S. Pat. No. 5,567,793. This reference also discloses the use of urethane-free, allophanate-modified isocyanates in RIM processes to improve the flex modulus of the resultant elastomers.

U.S. Pat. No. 5,663,272 discloses allophanate-modified MDI prepolymers and their use in RIM processes to improve the flex modulus of the resultant elastomers. These allophanate modified MDI's are made from a monoisocyanate with an organic compound having at least two OH groups to form a urethane, and then converting the urethane to allophanate by reacting with an isomeric mixture of MDI.

Allophanate-modified MDI's, various prepolymers thereof and other modifications thereof, can be used in various end use applications such as, for example, footwear and flexible foams, as is known and described in, for example, U.S. Pat. Nos. 5,663,272, 5,821,275 5,874,485 and 6,271,279.

Advantages of the present invention provides novel allophanate-modified diphenymethane diisocyanates that are storage-stable liquids at room temperature. These allophanates are more versatile in that they contain reactive unsaturation that allows dual reaction mechanisms, i.e. isocyanate-based and free radical based mechanisms. Such diphenylmethane diisocyanates are suitable for virtually any polyurethane process, such as urethane and/or urea foam, elastomers, thermoplastic urethanes, etc. In addition, they can be used in free radical-type reactions such as those involving radical polymerization of reactive monomers such as styrene, acrylonitrile, methyl methacrylate, etc.

### SUMMARY OF THE INVENTION

This invention relates to a liquid, storage-stable, allophanate-modified diisocyanate having an NCO group content of about 9.5 to about 32.3% by weight. This allophanate-modified diphenylmethane diisocyanate comprises the reaction product of:
(A) diphenylmethane diisocyanate which comprises:
   (1) from 0 to 6% by weight of the 2,2′-isomer,
   (2) from 0 to 76% by weight of the 2,4′-isomer, and
   (3) from 24 to 100% by weight of 4,4′-isomer,
   wherein the sum of the %'s by weight of (1), (2) and (3) totals 100% by weight of (A) the diphenylmethane diisocyanate; and
(B) at least one ethylenically unsaturated alcohol compound, in the presence of:
(C) at least one radical inhibitor which is free of NCO-reactive groups, and
(D) at least one allophanate catalyst.

In addition, the present invention relates to a process for the preparation of an allophanate-modified diphenylmethane diisocyanate. This process comprises (I) reacting (A) diphenylmethane diisocyanate comprising: (1) from 0 to 6% by weight of the 2,2′-isomer, (2) from 0 to 76% by weight of the 2,4′-isomer, and (3) from 24 to 100% by weight of 4,4′-isomer, wherein the sum of the %'s by weight of (1), (2) and (3) totals 100% by weight of (A) the diphenylmethane diisocyanate; and (B) at least one ethylenically unsaturated alcohol compound, in the presence of: (C) at least one radical inhibitor which is free of NCO-reactive groups, and (D) at least one allophanate catalyst, and (II) adding (E) a catalyst stopper, to the reaction mixture in (I) once the allophanate-modified isocyanate has the desired NCO group content.

### DETAILED DESCRIPTION OF THE INVENTION

As used in the present invention, the term liquid means that the allophanate-modified diiphenylmethane disocyanate product does not precipitate solids when stored at 25° C. for 3 months.

As used herein, the term “storage-stable” means that the allophanate-modified diphenylmethane diisocyanate product has up to a 1% absolute change in the % NCO group content and up to a 10% change in the viscosity when stored at 25° C. for 3 months.

The liquid, storage-stable, allophanate-modified diisocyanates of the present invention are typically characterized by an NCO group content of at least about 9.5% NCO, and preferably of at least about 15% NCO, and more preferably of at least about 19% NCO. These liquid diisocyanates are also typically characaterized by an NCO group content or less than or equal to about 32.3% NCO, preferably less than or equal to about 29% NCO and more preferably less than or equal to about 26% NCO. The liquid modified diisocyanates may also have an NCO group content ranging between any combination of these upper and lower values, inclusive. For example, the liquid diisocyanates may have an NCO group content of from about 9.5% by weight NCO to about 32.3% by weight NCO, preferably from about 15% by weight NCO to about 29% by weight NCO and more preferably from about 19% by weight NCO to about 26% by weight NCO.

In accordance with the present invention, suitable diisocyanates to be used as component (A) in the allophanate-modified diisocyanates of the invention comprise diphenylmethane diisocyanate in which the 2,2′-isomer is present in an amount of from 0 to 6% by weight, preferably 0 to 3% by weight, and more preferably 0 to 1% by weight; the 2,4′-isomer is present in an amount of 0 to 76% by weight, preferably 0 to 17% by weight, and more preferably 0 to 5% by weight; and the 4,4′-isomer is present in an amount of from 24 to 100% by weight, preferably 80 to 100% by weight, and more preferably 94 to 100% by weight. When mixtures of the 2,2′-isomer, the 2,4′-isomer and the 4,4′-isomer of diphenylmethane diisocyanate are used, the sum of the %'s by weight of the individual isomers totals 100% by weight of the diphenylmethane diisocyanate.

Suitable ethylenically unsaturated alcohols to be used as component (B) in the allophanate-modified diisocyanates of the present invention include, for example, compounds which contain at least one, and preferably only one α,β-ethylenically unsaturated group and one hydroxyl group. Suitable compounds to be used as the ethylenically unsaturated alcohols include those such as, for example, hydroxyalkyl acrylates, hydroxyalkyl methacrylates, hydroxyalkoxy acrylates, hydroxyalkoxy methacrylates, hydroxyaryl acrylates, hydroxyaryl methacrylates, aromatically-substituted ethylenically unsaturated monols, isopropenyl-phenyl monols, hydroxyl nitriles, etc. Some specific examples of such compounds for component (B) herein include 2-hydroxyethyl acrylate, 2-hydroxyethyl methacrylate, 2-hydroxypropyl acrylate, 2-hydroxypropyl methacrylate, 2-hydroxybutyl acrylate, 2-hydroxybutyl methacrylate, 2-hydroxypentyl acrylate, 2-hydroxypentyl methacrylate, 2-hydroxyhexyl acrylate, 2-hydroxyhexyl methacrylate, 2-hydroxyoctyl acrylate, 2-hydroxyoctyl methacrylate, diethylene glycol monoacrylate, diethylene glycol monomethacrylate, dipropylene glycol monoacrylate, dipropylene glycol monomethacrylate, 4-hydroxyphenyl acrylate, 4-hydroxyphenyl methacrylate, 2-hydroxyphenyl acrylate, 2-hydroxyphenyl methacrylate, 3-hydroxyphenyl acrylate, 3-hydroxyphenyl methacrylate, cinnamyl alcohol, isopropenylphenol, isopropenylbenzyl alcohol, α,α-dimethyl-m-isopropenylbenzyl alcohols, 4-hydroxycrotononitrile, etc. It is preferred that these ethylenically unsaturated alcohols have a molecular weight (number average) of from about 69 to about 1500. Preferred ethylenically unsaturated alcohols to be used as component (B) herein are 2-hydroxyethyl methacrylate, 2-hydroxylethyl acrylate, 2-hydroxylpropyl methacrylate, and cinnamyl alcohol.

Suitable compounds to be used as component (C), the radical inhibitor, in accordance with the present invention include, for example, any compound known to be capable of inhibiting the homopolymerization of compounds containing reactive unsaturation. Generally, such radical inhibitors include compounds which are free of groups that are capable of reacting with the NCO groups of the diisocyanate component. Some examples of suitable radical inhibitors to be used as component (C) in the present invention include 1,4-benzoquinone, phenothiazine, etc.

Suitable allophanate catalyst to be used as component (D) in accordance with the present invention include any known catalyst which is capable of promoting the allophanate reaction between a diisocyanate component and a component which contains one alcohol group. Some examples of such allophanate catalysts include, for example, zinc acetylacetonate (ZnAcAc), stannous octoate, zinc octoate, etc. A preferred catalyst is zinc acetylacetonate (ZnAcAc). In addition, the catalyst is typically present in an amount ranging from 25 to 200 ppm, based on the weight of (A) the isocyanate component.

The allophanate modified diphenylmethane diisocyanates of the present invention are prepared in accordance with known processes and procedures. Such processes include, for example, those described in U.S. Pat. Nos. 5,319,053, 5,319,054, 5,610,260, and U.S. Published application 20060084776, the disclosures of which are hereby incorporated by reference.

Suitable catalyst stoppers to be used in the process of the present invention include compounds such as, for example, benzoyl chloride, hydrochloric acid, di(2-ethylhexyl) phosphate, etc. Benzoyl chloride is a preferred stopper. When present, the stopper is typically added in an amount that is equal to about twice the weight of the allophanate catalyst.

The liquid, allophanate-modified isocyanates containing reactive unsaturation are, of course, suitable for the preparation of polyurethanes by known processes as described in the art. These liquid allophanate-modified isocyanates which contain reactive unsaturation also offer the advantages of increased versatility in that they contain reactive unsaturation which allows dual reaction mechanisms i.e. isocyanate-based and free radical based mechanisms. In addition, the liquid, allophanate-modified isocyanates described herein may be used to prepare novel macromers which contain ethylenic unsaturation, and are thus, useful in the preparation of novel preformed stabilizers and in the preparation of novel polymer polyols. These macromers and other related products are not the subject of the present application, but are described in commonly assigned U.S. application Ser. No. ______, (Agent Docket Number PO-8911/MD-05-083) filed in the United States Patent and Trademark Office on Dec. ______, 2006, i.e. the same day as the present application.

The following examples further illustrate details for the preparation and use of the compositions of this invention. The invention, which is set forth in the foregoing disclosure, is not to be limited either in spirit or scope by these examples. Those skilled in the art will readily understand that known variations of the conditions and processes of the following preparative procedures can be used to prepare these compositions. Unless otherwise noted, all temperatures are degrees Celsius and all parts and percentages are parts by weight and percentages by weight, respectively.

### EXAMPLES

The following components were used in the working examples:
Isocyanate A: 4,4-diphenylmethane diisocyanate having an NCO group content of about 33.6%
HEMA: 2-hydroxyethyl methacrylate, an ethylenically unsaturated alcohol
Inhibitor A: 2,6-tert-butyl-4-methylphenol (commonly referred to as BHT)
Inhibitor B: 1,4-benzoquinone
Catalyst A: zinc acetylacetonate (ZnAcAc)
Catalyst Stopper: benzoyl chloride

### Example 1

### This Example Illustrates Importance of Proper Inhibitors for a Stable Liquid MDI Allophanate

2-Hydroxyethyl methacrylate (HEMA, 28 g) was added to a mixture of Isocyanate A (301.7 g) and Inhibitor A (0.5 g) at 60° C. under nitrogen. The solution was allowed to exotherm to 86° C. and stirred for 30 minutes. Catalyst A (Zn AcAc, 0.056 g) was added to the mixture, followed by heating at 90° C. for 1 hour. The solution was cooled to 65° C., and Catalyst Stopper (0.13 g) was added to deactivate the catalyst. A light yellow liquid was obtained which had an NCO group content of 27.2%. Upon standing overnight, the resultant product solidified.

### Example 2

HEMA (20 g) was added to a mixture of Isocyanate A (250 g) and Inhibitor B (0.08 g) at 60° C. under nitrogen. The solution was heated at 60° C. for 75 minutes. Catalyst A (0.04 g) was added and the reaction solution was heated at 90° C. for 75 minutes. After cooling to 60° C., Catalyst Stopper (0.08 g) was added to yield a yellow liquid product which had an NCO group content of 26%. This product remained liquid for over 15 months, with no visible solids formation.

### Example 3

HEMA (30 g) was added to a mixture of Isocyanate A (250 g) and Inhibitor B (0.16 g) at 60° C. under nitrogen. The solution was heated at 60° C. for 75 minutes. Catalyst A (0.04 g) was added and the reaction solution was heated at 90° C. for 75 minutes. After cooling to 60° C., the Catalyst Stopper (0.08 g) was added to yield a storage stable yellow liquid having an NCO group content of 22.6%. This product remained liquid for over 15 months, with no visible solids formation.

### Example 4

HEMA (80 g) was added to a mixture of Isocyanate A (500 g) and Inhibitor B (0.4 g) at 60° C. under nitrogen. The solution was heated at 60° C. for 75 minutes. Catalyst A (0.08 g) was added and the reaction solution was heated at 90° C. for 75 minutes. After cooling to 60° C., the Catalyst Stopper (0.16 g) was added to give a storage stable yellow liquid having an NCO group content of 20.0%.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A liquid, storage-stable, allophanate-modified diisocyanate having an NCO group content of about 9.5 to about 32.3% by weight, and comprising the reaction product of:
(A) diphenylmethane diisocyanate which comprises:
(1) from 0 to 6% by weight of the 2,2′-isomer,
(2) from 0 to 76% by weight of the 2,4′-isomer, and
(3) from 24 to 100% by weight of 4,4′-isomer,
wherein the sum of the %'s by weight of (1), (2) and (3) totals 100% by weight of (A) the diphenylmethane diisocyanate; and
(B) at least one ethylenically unsaturated alcohol compound, in the presence of:
(C) at least one radical inhibitor which is free of NCO-reactive groups, and
(D) at least one allophanate catalyst.

2. The liquid, storage-stable, allophanate-modified diisocyanate of claim 1, which is characterized by an NCO group content of from about 15 to about 29% by weight.

3. The liquid, storage-stable, allophanate-modified diisocyanate of claim 1, wherein component (A) comprises diphenylmethane diisocyanate which comprises (1) from 0 to 3% by weight of the 2,2′-isomer, (2) from 0 to 17% by weight of the 2,4′-isomer, and (3) from 80 to 100% by weight of 4,4′-isomer, with the sum of the %'s by weight of (1), (2) and (3) totaling 100% by weight of (A) the diphenylmethane diisocyanate.

4. The liquid, storage-stable, allophanate-modified diisocyanate of claim 1, wherein (B) said ethylenically unsaturated alcohol is selected from the group consisting of hydroxyalkyl acrylates, hydroxylalkyl methacrylates, hydroxylalkoxy acrylates, hydroxylalkoxy methacrylates, hydroxylaryl acrylates, hydroxyaryl methacrylates, aromatic-substituted ethylenically unsaturated monols, isopropenylphenyl monols, hydroxyl nitriles and mixtures thereof.

5. The liquid, storage-stable, allophanate-modified diisocyanate of claim 1, wherein (C) said inhibitor is selected from the group consisting of 1,4-benzoquinone, phenothiazine and mixtures thereof.

6. The liquid, storage-stable, allophanate-modified diisocyanate of claim 1, wherein (D) said allophanate catalyst is selected from the group consisting of zinc acetylacetonate, stannous octoate, zinc octoate and mixtures thereof.

7. The liquid, storage-stable, allophanate-modified diisocyanate of claim 3, wherein component (A) comprises diphenylmethane diisocyanate which comprises (1) from 0 to 1% by weight of the 2,2′-isomer, (2) from 0 to 5% by weight of the 2,4′-isomer, and (3) from 94 to 100% by weight of 4,4′-isomer, with the sum of the %'s by weight of (1), (2) and (3) totaling 100% by weight of (A) the diphenylmethane diisocyanate.

8. The liquid, storage-stable, allophanate-modified diisocyanate of claim 1, wherein (B) said ethylenically unsaturated alcohol compound has a molecular weight in the range of from 69 to 1500.

9. A process for the preparation of a liquid, storage-stable, allophanate-modified diisocyanate having an NCO group content of about 9.5% to about 32.3% by weight, comprising:
(I) reacting
(A) diphenylmethane diisocyanate comprising:
(1) from 0 to 6% by weight of the 2,2′-isomer,
(2) from 0 to 76% by weight of the 2,4′-isomer, and
(3) from 24 to 100% by weight of 4,4′-isomer,
wherein the sum of the %'s by weight of (1), (2) and (3) totals 100% by weight of (A) the diphenylmethane diisocyanate; and
(B) at least one ethylenically unsaturated alcohol compound, in the presence of:
(C) at least one radical inhibitor which is free of NCO-reactive groups, and
(D) at least one allophanate catalyst, and
(II) adding
(E) a catalyst stopper,
to the reaction mixture in (I) once the allophanate-modified isocyanate has the desired NCO group content.

10. The process of claim 9, wherein the liquid, storage-stable, allophanate-modified diisocyanate has an NCO group content of from about 15 to about 29% by weight.

11. The process of claim 9, wherein component (A) comprises diphenylmethane diisocyanate which comprises (1) from 0 to 3% by weight of the 2,2′-isomer, (2) from 0 to 17% by weight of the 2,4′-isomer, and (3) from 80 to 100% by weight of 4,4′-isomer, with the sum of the %'s by weight of (1), (2) and (3) totaling 100% by weight of (A) the diphenylmethane diisocyanate.

12. The process of claim 9, wherein (B) said ethylenically unsaturated alcohol is selected from the group consisting of hydroxyalkyl acrylates, hydroxylalkyl methacrylates, hydroxylalkoxyl acrylates, hydroxylalkoxy methacrylates, hydroxylaryl acrylates, hydroxyaryl methacrylates, aromatic-substituted ethylenically unsaturated monols, isopropenylphenyl monols, hydroxyl nitriles and mixtures thereof.

13. The process of claim 9, wherein (C) said inhibitor is selected from the group consisting of 1,4-benzoquinone, phenothiazine and mixtures thereof.

14. The process of claim 9, wherein (D) said allophanate catalyst is selected from the group consisting of zinc acetylacetonate, stannous octoate, zinc octoate and mixtures thereof.

15. The process of claim 9, wherein (E) said stopper comprises an acidic additive.

16. The process of claim 11, wherein component (A) comprises diphenylmethane diisocyanate which comprises diphenylmethane diisocyanate which comprises (1) from 0 to 1% by weight of the 2,2′-isomer, (2) from 0 to 5% by weight of the 2,4′-isomer, and (3) from 94 to 100% by weight of 4,4′-isomer, with the sum of the %'s by weight of (1), (2) and (3) totaling 100% by weight of (A) the diphenylmethane diisocyanate.

17. The process of claim 9, wherein (B) said ethylenically unsaturated alcohol compound has a molecular weight in the range of from 69 to 1500.
